(19) United States
(12) Patent Application Publication
(10) Pub. No.: US 2026/0294401 A1
(43) Pub. Date: Oct. 1, 2026

(54) ANALYSIS APPARATUS, ULTRASOUND DIAGNOSTIC APPARATUS, AND STORAGE MEDIUM

(71) Applicant: CANON MEDICAL SYSTEMS CORPORATION, Tochigi (JP)

(72) Inventors: Chihiro SHIBATA, Nasushiobara (JP); Kuramitsu NISHIHARA, Otawara (JP)

(73) Assignee: CANON MEDICAL SYSTEMS CORPORATION, Tochigi (JP)

(21) Appl. No.: 19/572,341

(22) Filed: Mar. 19, 2026

(30) Foreign Application Priority Data
Mar. 27, 2025 (JP) 2025-052992

(51) Int. Cl. A61B 8/00

(52) U.S. Cl. CPC A61B 8/5207; A61B 8/462

(57) ABSTRACT

An analysis apparatus according to an embodiment includes processing circuitry. The processing circuitry calculates a parameter related to elasticity using data obtained by scanning of a subject. The processing circuitry determines, on the basis of a first determination condition related to the data and the parameter, a display region and a non-display region for an image based on the parameter related to elasticity, in a scan region where the scanning has been executed. The processing circuitry determines, on the basis of a second determination condition having a determination criterion higher than that of the first determination condition with respect to the data and the parameter, a generation condition for the image. The processing circuitry causes an image to be displayed, the image having been generated on the basis of the generation condition for the display region in the scan region.

### CROSS-REFERENCE TO RELATED APPLICATIONS

This application is based upon and claims the benefit of priority from Japanese Patent Application No. 2025-052992, filed on Mar. 27, 2025; the entire contents of which are incorporated herein by reference.

### FIELD

Embodiments described herein relate generally to an analysis apparatus, an ultrasound diagnostic apparatus, and a storage medium.

### BACKGROUND

In scanning performed in a strain elastography mode by an ultrasound diagnostic apparatus, an ultrasound probe is pressed against a living body to artificially generate slight vibration, displacement of each tissue detected on the basis of that vibration is differentiated to calculate strain values, and elasticity of the tissue is visually displayed by performing coloring according to the strain values.

In the strain elastography mode, strain values are obtained at all sample points within a specified ROI or over the entire scan region, and coloring can be performed such that, for example, the average of the strain values is displayed in green, any strain value higher than the average (softer part) is displayed in red, and any strain value lower than the average (harder part) is displayed in blue.

Tumors are generally hard and are known to be displayed in blue in the above mentioned coloring example. Furthermore, within the ROI, there may be, not only biological signals, but also regions not suitable for calculating strain values, such as noise regions, blood flow regions having fluidity, and regions affected by pulsation.

### BRIEF DESCRIPTION OF THE DRAWINGS

FIG. 1 is a block diagram illustrating an example of an overall configuration of an ultrasound diagnostic apparatus according to a first embodiment;

FIG. 2 is a diagram illustrating an example of display in a strain elastography mode;

FIG. 3 is a diagram illustrating an example of a configuration of Doppler processing circuitry and image processing circuitry, according to the first embodiment;

FIG. 4 is a diagram illustrating an example of display according to the first embodiment;

FIG. 5 is a flowchart illustrating a procedure of processing of the ultrasound diagnostic apparatus according to the first embodiment; and

FIG. 6 is a block diagram illustrating an example of a configuration of an analysis apparatus according to another embodiment.

### DETAILED DESCRIPTION

An analysis apparatus according to an embodiment includes processing circuitry. The processing circuitry is configured to calculate a parameter related to elasticity using data obtained by scanning of a subject. The processing circuitry is configured to determine, on the basis of a first determination condition related to the data and the parameter, a display region and a non-display region for an image based on the parameter related to elasticity, in a scan region where the scanning has been executed. The processing circuitry is configured to determine, on the basis of a second determination condition having a determination criterion higher than that of the first determination condition with respect to the data and the parameter, a generation condition for the image. The processing circuitry is configured to cause an image to be displayed, the image having been generated on the basis of the generation condition for the display region in the scan region.

Embodiments of an analysis apparatus, an ultrasound diagnostic apparatus, and a program, according to the present application, will hereinafter be described in detail by reference to the appended drawings. The analysis apparatus, the ultrasound diagnostic apparatus, and the program, according to the present application, are not to be limited by the following embodiments.

### First Embodiment

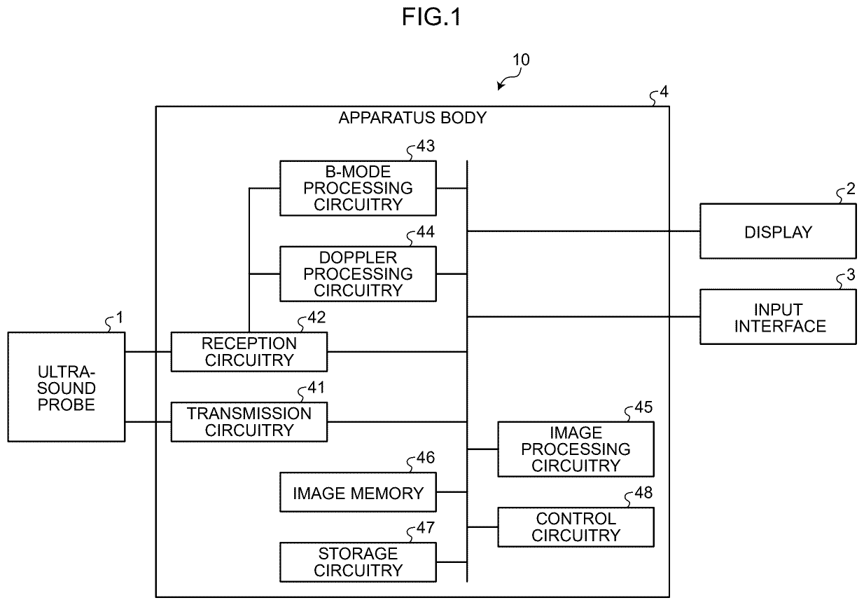

FIG. 1 is a block diagram illustrating an example of an overall configuration of an ultrasound diagnostic apparatus 10 according to a first embodiment. As illustrated in FIG. 1, the ultrasound diagnostic apparatus 10 according to the first embodiment has an ultrasound probe 1, a display 2, an input interface 3, and an apparatus body 4. The ultrasound probe 1, the display 2, and the input interface 3 are communicably connected to the apparatus body 4.

The ultrasound probe 1 has a plurality of piezoelectric transducers, and these plurality of piezoelectric transducers generate ultrasound on the basis of a driving signal supplied from transmission circuitry 41. Furthermore, the ultrasound probe 1 receives reflected waves from a subject and converts the reflected waves into an electric signal. The ultrasound probe 1 also has, for example, matching layers provided on the piezoelectric transducers, and a backing material that prevents propagation of ultrasound backward from the piezoelectric transducers. The ultrasound probe 1 is detachably connected to the apparatus body 4.

When ultrasound is transmitted from the ultrasound probe 1 to a subject, the ultrasound transmitted is successively reflected by acoustic impedance discontinuities in tissue in the body of the subject, and is received as a reflected wave signal by the plurality of piezoelectric transducers included in the ultrasound probe 1. Amplitude of the reflected wave signal received is dependent on the acoustic impedance differences at the discontinuities where the ultrasound is reflected. When the transmitted ultrasonic pulses are reflected by, for example, blood flow or a surface of a cardiac wall, which is moving, frequency of the reflected wave signal is shifted dependently on a velocity component of that moving object in relation to the direction in which the ultrasound is transmitted, due to the Doppler effect.

The ultrasound probe 1 may be a one-dimensional ultrasound probe having a plurality of piezoelectric transducers arranged in a row, an ultrasound probe that mechanically oscillates a plurality of piezoelectric transducers of a one-dimensional ultrasound probe, or a two-dimensional ultrasound probe having a plurality of piezoelectric transducers arranged two-dimensionally in a grid pattern.

The display 2 displays a graphical user interface (GUI) for an operator of the ultrasound diagnostic apparatus 10 to input various setting requests using the input interface 3, and displays, for example, an ultrasound image generated at the apparatus body 4. The display 2 also displays various messages and display information to notify the operator of processing statuses of the apparatus body 4 and processing results. Furthermore, the display 2 has a speaker and is capable of outputting sound.

The input interface 3 is operated for performing various setting, for example, and is implemented by any of, for example, a trackball, a switch button, a mouse, a keyboard, a touch pad with an operation surface touched for input operation, a touch monitor having a display screen and a touch pad integrated together, a non-contact input circuit using an optical sensor, and a sound input circuit. The input interface 3 is connected to control circuitry 48 described later and converts input operation received from an operator into an electric signal and outputs the electric signal to the control circuitry 48. According to this specification, the input interface 3 is not necessarily an input interface including physical operation parts, such as a mouse and a keyboard. For example, examples of the input interface 3 also include a processing circuit that receives an electric signal corresponding to input operation from an external input device provided separately from the apparatus and outputs this electric signal to the control circuitry 48.

The apparatus body 4 is an apparatus that generates an ultrasound image on the basis of a reflected wave signal received by the ultrasound probe 1. As illustrated in FIG. 1, the apparatus body 4 has transmission circuitry 41, reception circuitry 42, B-mode processing circuitry 43, Doppler processing circuitry 44, image processing circuitry 45, an image memory 46, storage circuitry 47, and control circuitry 48. The transmission circuitry 41, the reception circuitry 42, the B-mode processing circuitry 43, the Doppler processing circuitry 44, the image processing circuitry 45, the image memory 46, the storage circuitry 47, and the control circuitry 48 are communicably connected to one another. Processing functions in the form of programs executable by a computer have been stored in the storage circuitry 47 in the ultrasound diagnostic apparatus 10 illustrated in FIG. 1. The transmission circuitry 41, the reception circuitry 42, the B-mode processing circuitry 43, the Doppler processing circuitry 44, the image processing circuitry 45, and the control circuitry 48 are processors that implement functions corresponding to the respective programs by reading the programs from the storage circuitry 47 and executing the read programs. In other words, each of the circuitry that have read the respective programs has the function corresponding to the read program.

The transmission circuitry 41 controls transmission of ultrasound by the ultrasound probe 1. For example, on the basis of an instruction from the control circuitry 48 described later, the transmission circuitry 41 applies a driving signal (driving pulse) to the ultrasound probe 1 according to timing having a predetermined transmission delay time assigned to each transducer. The transmission circuitry 41 thereby causes an ultrasound beam to be transmitted, the ultrasound beam having ultrasound focused into a beam shape. The reception circuitry 42 controls reception of a reflected wave signal resulting from reflection of transmitted ultrasound by tissue in the body. For example, on the basis of an instruction from the control circuitry 48 described later, the reception circuitry 42 performs addition processing by giving a predetermined delay time to the reflected-wave signal received by the ultrasound probe 1. A reflected component from a direction corresponding to reception directivity of the reflected-wave signal is thereby emphasized. The reception circuitry 42 then converts the reflected wave signal that has been subjected to the addition processing, into an in-phase signal (I signal) and a quadrature-phase signal (Q signal) of a baseband. The reception circuitry 42 then transmits the I signal and the Q signal (hereinafter, referred to as the IQ signal) as reflected wave data, to the B-mode processing circuitry 43 and the Doppler processing circuitry 44. The reception circuitry 42 may convert the reflected wave signal that has been subjected to the addition processing into a radio frequency (RF) signal and transmit the RF signal to the B-mode processing circuitry 43 and the Doppler processing circuitry 44. The IQ signal and the RF signal are signals (reflected wave data) including phase information.

The B-mode processing circuitry 43 performs, for example, logarithmic amplification and envelope detection processing on the reflected-wave data to generate data (B-mode data) including luminance levels respectively representing signal intensities at a plurality of sample points (observation points). The B-mode processing circuitry 43 transmits the B-mode data generated, to the image processing circuitry 45.

By performing frequency analysis on the reflected wave data to obtain velocity information, the Doppler processing circuitry 44 generates data (Doppler data) including motion information extracted for each sample point, the motion information being based on the Doppler effect of the moving object within the scan range. Specifically, the Doppler processing circuitry 44 generates Doppler data including, as the motion information of the moving object, an average velocity, a variance value, and a power value, extracted for each of the plurality of sample points. The moving body herein is, for example, blood flow, tissue, such as a cardiac wall, or a contrast agent. The Doppler processing circuitry 44 according to the embodiment generates, as motion information of blood flow (blood flow information), estimated information on an average velocity of the blood flow, an average variance value of the blood flow, and an average power value of the blood flow for each of the plurality of sample points, for example. That is, the blood flow information is information including a value based on the blood flow at each sample point (a value representing the blood flow).

For example, the Doppler processing circuitry 44 calculates the blood flow information by a color Doppler method. In the color Doppler method, ultrasound transmission and reception are performed multiple times along the same scan line, an MTI filter is applied to a data sequence at the same position, any signal originating from stationary tissue or slowly moving tissue (clutter signals) is minimized, and a signal originating from the blood flow is extracted. In the color Doppler method, pieces of blood flow information, such as a velocity of the blood flow, variance of the blood flow, and power of the blood flow, are estimated from this blood flow signal.

Furthermore, the Doppler processing circuitry 44 calculates information related to motion of tissue by a tissue Doppler imaging (TDI) method for displaying a spatial distribution of information related to motion of tissue. The Doppler processing circuitry 44 is able to generate elasticity information representing elasticity of the tissue by applying the information related to the motion of the tissue obtained by the TDI method. The Doppler processing circuitry 44 is an example of processing circuitry.

The image processing circuitry 45 performs, for example, processing to generate image data (ultrasound image data) and various image processing of the image data. For example, the image processing circuitry 45 converts scanning formats of B-mode data (morphological information) generated by the B-mode processing circuitry 43 and blood flow information and elasticity information generated by the Doppler processing circuitry 44 into a data format for display (scan conversion). The image processing circuitry 45 thereby generates each of B-mode image data (morphological image data) representing morphology of a structure of a subject, blood flow image data representing motion of blood flow in the subject, and elasticity image data representing tissue elasticity in the subject.

Furthermore, the image processing circuitry 45 performs, as various image processing other than the scan conversion, for example, image processing (smoothing processing) for regenerating an average luminance image using a plurality of image frames that have been scan-converted, and image processing (edge enhancement processing) using a differential filter in an image. Furthermore, the image processing circuitry 45 composites, for example, character information of various parameters, scales, and body marks onto an ultrasound image.

Furthermore, in a case where three-dimensional data (three-dimensional B-mode data and three-dimensional Doppler data) have been generated, the image processing circuitry 45 generates volume data by performing coordinate transformation according to an ultrasound scanning mode of the ultrasound probe 1. The image processing circuitry 45 then generates two-dimensional image data for display, by performing various rendering processes on the volume data.

The image processing circuitry 45 stores image data generated and image data that have been subjected to various image processing, into the image memory 46. The image processing circuitry 45 may generate, with the image data, ancillary information related to diagnosis, including information indicating display positions of respective sets of image data, various information for assisting in operation of the ultrasound diagnostic apparatus, and patient information, and store them into the image memory 46.

The image memory 46 is a memory that stores image data (such as B-mode image data, blood flow image data, and elasticity image data) generated by the image processing circuitry 45. Furthermore, the image memory 46 is also capable of storing data generated by the B-mode processing circuitry 43 and the Doppler processing circuitry 44. For example, the B-mode data, blood flow information, and elasticity information stored in the image memory 46 are able to be called by an operator after diagnosis, and serve as ultrasound image data for display via the image processing circuitry 45.

The storage circuitry 47 stores a control program for performing ultrasound transmission and reception, image processing, and display processing, as well as various data, such as diagnostic information (for example, patient IDs, and findings of physicians), diagnostic protocols, and various body marks. Furthermore, the storage circuitry 47 is used, as required, for storage of image data stored in the image memory 46, for example. Furthermore, data stored in the storage circuitry 47 may be transferred to an external device via a communication interface not illustrated in the drawings.

The control circuitry 48 controls the overall processing of the ultrasound diagnostic apparatus 10. Specifically, on the basis of various setting requests input by an operator via the input interface 3, and various control programs and various data read from the storage circuitry 47, the control circuitry 48 controls processing of, for example, the transmission circuitry 41, the reception circuitry 42, the B-mode processing circuitry 43, the Doppler processing circuitry 44, and the image processing circuitry 45. Furthermore, the control circuitry 48 causes the display 2 to display ultrasound image data stored in the image memory 46. The control circuitry 48 is an example of processing circuitry.

An example of the overall configuration of the ultrasound diagnostic apparatus 10 according to the first embodiment has been described above. The ultrasound diagnostic apparatus 10 configured as described above enables color display based on appropriate strain values for a region to be observed within an image. As described above, in the strain elastography mode for displaying elasticity of tissue, strain values are calculated by differentiating displacement of each tissue detected on the basis of slight vibration artificially generated, and coloring according to the strain values is performed, but in a case where colors are set using strain values in a region not suitable for strain value calculation, coloring may be not performed appropriately for the target to be observed.

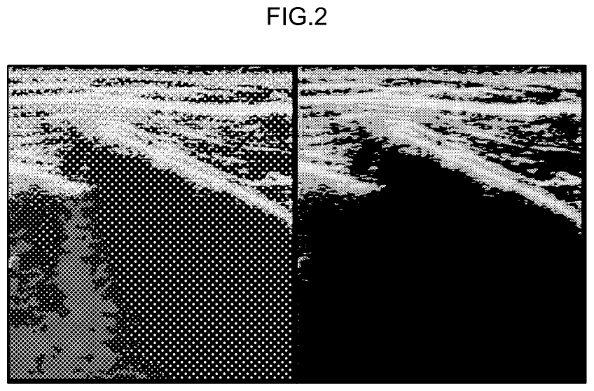

FIG. 2 is a diagram illustrating an example of display in the strain elastography mode. In FIG. 2, a B-mode image is illustrated on the right and an example of an elasticity image in a case where coloring according to a strain value is performed for each position in the B-mode image is illustrated on the left. Furthermore, in FIG. 2, an example where colors are set using strain values at all of positions on the B-mode image is illustrated. Furthermore, tissue illustrated in the image in FIG. 2 includes a region largely affected by pulsation in the lower left of the image. In a case where an elasticity image having colors set using strain values of all of positions is displayed for such tissue, strain values in the lower left region of the image are extremely high and affect the average value and the maximum value of the strain values. That is, as illustrated in FIG. 2, the lower left region in the image is displayed in a color representing a soft part (for example, red) or in a color corresponding to the average strain value (for example, green). In contrast, because tissue to be actually observed (the upper region in the image) has lower strain values than the lower left region in the image, the entire region is displayed in a color representing a hard part for example, blue) and thus differences in hardness within this region cannot be recognized.

As described above, in the strain elastography mode, tissue scanned may include a region not suitable for calculation of stain values (calculation of the average value and maximum value of strain values) but tissue characteristics are determined on the basis of the pattern of the coloring even for such a region and the region thus needs to be a target to be observed. For example, a bag-shaped cyst filled with liquid is displayed in a blue-green-red pattern (BGR pattern) from a side closer to the body surface, and the presence of the cyst can be determined from this pattern.

The ultrasound diagnostic apparatus 10 according to the embodiment thus enables color display based on appropriate strain values for a region to be observed in an image by determining each of the region to be observed (a display region of an elasticity image) and a region to be subjected to strain value calculation (calculation of the average value and maximum value of strain values) from data collected in the strain elastography mode, determining an image condition for the elasticity image using strain values of the region to be subjected to the strain value calculation, and displaying the region to be observed in color under the image condition determined.

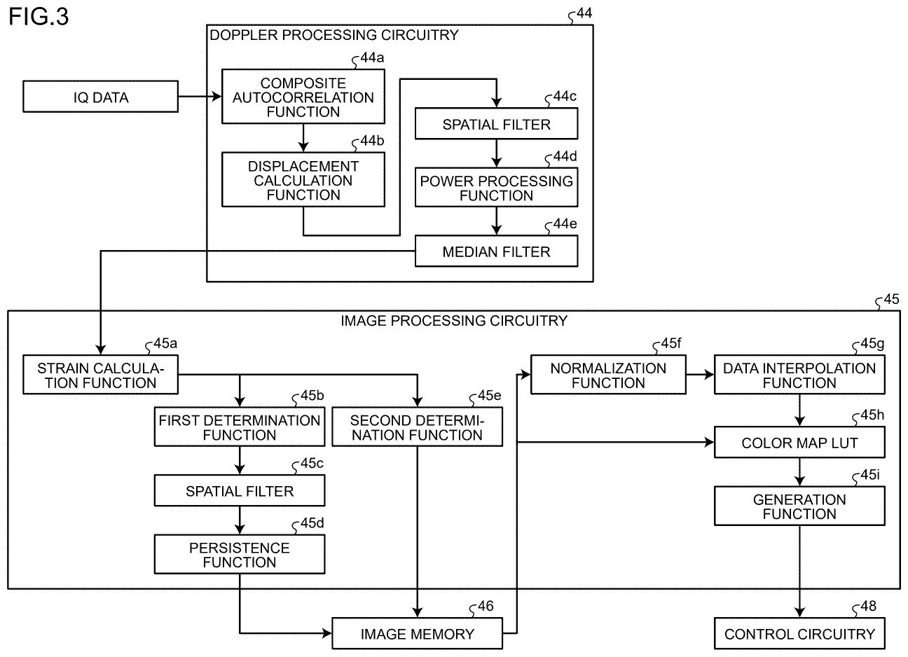

FIG. 3 is a diagram illustrating an example of a configuration of the Doppler processing circuitry 44 and the image processing circuitry 45, according to the first embodiment. As illustrated in FIG. 3, the Doppler processing circuitry 44 has a composite autocorrelation function 44a, a displacement calculation function 44b, a spatial filter 44c, a power processing function 44d, and a median filter 44e, and calculates a parameter related to elasticity using data obtained by scanning of a subject. The image processing circuitry 45 has a strain calculation function 45a, a first determination function 45b, a spatial filter 45c, a persistence function 45d, a second determination function 45e, a normalization function 45f, a data interpolation function 45g, a color map lookup table (LUT) 45h, and a generation function 45i. The image processing circuitry 45 is an example of processing circuitry.

The composite autocorrelation function 44a calculates a correlation between IQ data obtained before and after tissue compression in the strain elastography mode (a plurality of IQ signals obtained by pressing and releasing biological tissue from the body surface using the ultrasound probe 1) and outputs a position where the correlation is maximized, to the displacement calculation function 44b. The composite autocorrelation function 44a is capable of calculating the correlation between the IQ data by a composite autocorrelation method including a combination of an autocorrelation method and a cross-correlation method. The autocorrelation method is a method of estimating, from signals obtained before and after compression of tissue, a displacement distribution within the tissue generated by the compression, by using the Doppler principle used in blood flow measurement. The cross-correlation method is a method of estimating, from signals obtained before and after the compression of the tissue, a displacement distribution within the tissue generated by the compression, by template matching using a correlation function. By using the composite autocorrelation method, the composite autocorrelation function 44a is able to precisely estimate a displacement distribution even in a case where tissue has been deformed largely.

For example, when an IQ signal before compression is “X(t, y)”, and an IQ signal after the compression is “Y(t, y)”, a cross-correlation function “Rxy(t, y; m, n)” for these two IQ signals is defined by Equation 1 below.

$${R}_{xy} ( t , y ; m , n ) = {\sum }_{l = - L}^{L} {\sum }_{k = - K}^{K} X ( t + k , y + l ) {Y}^{*} ( t + m + k , y + n + l ) \qquad ( 1 )$$

In Equation 1, “t” represents a coordinate in a direction of an ultrasound beam (axial direction), “y” represents a coordinate in a direction orthogonal to the direction of the ultrasound beam (lateral direction), “m” represents a sampling position in the axial direction, and “n” represents a sampling position in the lateral direction. Furthermore, “1” represents a correlation window size in the axial direction and “k” represents a correlation window size in the lateral direction.

In the composite autocorrelation method, using the cross-correlation function expressed by Equation 1 above, for example, a correlation coefficient “C_{xy}(t, y; m, n)” is defined as expressed by Equation 2 below.

$${C}_{xy} ( t , y ; m , n ) = \frac{\vert {R}_{xy} ( t , y ; m , n ) \vert}{\sqrt{{R}_{xx} ( t , y ; 0 , 0 ) {R}_{yy} ( t , y ; m , n )}} \qquad ( 2 )$$

In Equation 2, “Rₓₓ(t, y; 0, 0)” represents an autocorrelation function related to the signal before the compression and “R_{yy}(t, y; m, n)” represents an autocorrelation function related to the signal after the compression. The composite autocorrelation function 44a calculates, for each measurement point (t, y), a correlation coefficient of “Equation 2” and a phase difference “ø (t, y; m, n)” of the cross-correlation function of “Equation 1” expressed by Equation 3 below, for all “m” and “n”.

$$ϕ ( t , y ; m , n ) = angle \{ {R}_{xy} ( t , y ; m , n ) \} \qquad ( 3 )$$

On the basis of results of the calculation performed by the composite autocorrelation function 44a, the displacement calculation function 44b calculates displacement (displacement distribution) at each measurement point. For example, in a case where “m” and “n” where the correlation coefficient of Equation 2 is maximized are “mₘₐₓ” and “nₘₐₓ”, the displacement calculation function 44b calculates a displacement distribution defined by Equation 4 below.

$${U}_{x} ( x , y ) = \frac{{m}_{max}}{2} + \frac{ϕ ( t , {m}_{max} )}{2 π} \qquad ( 4 )$$

$${U}_{y} ( x , y ) = {n}_{max}$$

In Equation 4, “Uₓ(x, y)” represents a displacement distribution (unit: wavelength) in the axial direction and “U_{y}(x, y)” represents a displacement distribution (unit: scan line spacing) in the lateral direction.

The spatial filter 44c performs filtering processing, such as noise removal, on output of the displacement calculation function 44b. The power processing function 44d executes power processing for removing, from displacement output by the displacement calculation function 44b, displacement whose corresponding signal power value (signal intensity) is below a threshold. The median filter 44e performs smoothing processing on the displacement distribution that has been subjected to the power processing. The processing by the median filter 44e enables reduction of the influence of any error that occurs in the processing performed by the composite autocorrelation function 44a.

The Doppler processing circuitry 44 outputs processing results of the above described processing to the image processing circuitry 45. The Doppler processing circuitry 44 is also capable of calculating displacement at each measurement point, a correlation coefficient, and a variance value within a predetermined range, and outputting them to the image processing circuitry 45.

The strain calculation function 45a calculates a strain value (strain distribution) at each measurement point by spatially differentiating a displacement distribution input from the Doppler processing circuitry 44. For example, the strain calculation function 45a calculates a strain distribution on the basis of Equation 5 below.

$${E}_{x} ( x , y ) = \frac{\partial {U}_{x} ( x , y )}{\partial x} \qquad ( 5 )$$

$${E}_{y} ( x , y ) = \frac{\partial {U}_{y} ( x , y )}{\partial y}$$

In Equation 5, “Eₓ(x, y)” represents a strain distribution in the axial direction and “E_{y}(x, y)” represents a strain distribution in the lateral direction.

On the basis of a first determination condition related to data obtained by scanning of a subject and a parameter calculated by using the data, the first determination function 45b determines a display region and a non-display region for an image based on a parameter related to elasticity in a scan region where the scanning has been executed. Specifically, the first determination function 45b determines a display region for color display of strain elastography and a non-display region for no color display, in a scan region, on the basis of the first determination condition having a determination criterion related to signal intensities of data (power values of Doppler data) and a determination criterion related to at least one of displacement and variance of a part included in the scan region and a correlation coefficient related to pattern matching of the part.

That is, the first determination function 45b separates measurement points into the display region for color display and the non-display region, on the basis of a signal intensity at each measurement point of data collected in the strain elastography mode and displacement, variance, and a correlation coefficient at each measurement point calculated by the Doppler processing circuitry 44. For example, for each of the data and the parameter, the first determination condition has, as a determination criterion, a threshold based on an absolute value or a threshold based on a relative value. In this case, the first determination function 45b determines the display region and the non-display region by comparing thresholds set for the signal intensity, displacement, correlation coefficient, and variance (variance of displacement and variance of the correlation coefficient) with results at each measurement point. Variance values of the Doppler data may be used as the variance. A threshold equal to or less than a value reduced by α% from the maximum value, for example, may be set as the threshold based on the relative value.

In a case where signal intensities are used for determination, the first determination function 45b determines any measurement point having a signal intensity higher than the threshold as the display region and any measurement point having a signal intensity equal to or less than the threshold as the non-display region. Two thresholds (first threshold>second threshold) may be set for the signal intensities. In this case, the first determination function 45b may determine any measurement point having a signal intensity lower than the first threshold and higher than the second threshold as the display region and any other measurement point as the non-display region.

In a case where displacement is used for determination, the first determination function 45b determines any measurement point having displacement higher than the threshold as the non-display region and any measurement point having displacement equal to or less than the threshold as the display region. That is, the first determination function 45b determines a region having extremely high displacement as the non-display region.

In a case where variance is used for determination, the first determination function 45b determines any measurement point having displacement variance, correlation coefficient variance, or Doppler variance higher than the threshold as the non-display region, and determines any measurement point where that variance is equal to or less than the threshold as the display region.

In a case where correlation coefficients are used for determination, the first determination function 45b determines any measurement point having a correlation coefficient lower than the threshold (for example, 0.6) as the non-display region and any measurement point having a correlation coefficient larger than the threshold as the display region.

The first determination function 45b may determine the display region and the non-display region using one of the determination criteria described above, or may determine the display region and the non-display region using more than one of these determination criteria.

The first determination condition includes a determination criterion including a combination of the data and the parameter. Specifically, the combination of the data and the parameter includes at least one of an arithmetic operation using the data and the parameter and a logical operation using the data and the parameter. That is, the first determination function 45b is able to determine the display region and the non-display region by combining the signal intensities, displacement, variance, and correlation coefficients. For example, the first determination function 45b may calculate a new parameter by an arithmetic operation using at least two or more of the signal intensity, displacement, variance, and correlation coefficient (for example, multiplication of the signal intensity and the displacement) and determine the display region and the non-display region using the calculated new parameter.

Furthermore, the first determination condition may include a logical sum of the data and the parameter, and the first determination function 45b may determine a region satisfying the condition of the logical sum in the scan region as the display region of the image. That is, the first determination function 45b executes, for each measurement point, at least two or more determinations of the determination based on the signal intensity, the determination based on the displacement, the determination based on the variance, and the determination based on the correlation coefficient, and in a case where a determination result indicating that the measurement point corresponds to the display region is obtained in any of these determinations, the first determination function 45b determines that measurement point as the display region.

For example, the first determination function 45b calculates the logical sum of the determination based on the signal intensity and the determination based on the correlation coefficient, and classifies any measurement point determined as the display region in at least one of the determinations as the display region.

The classification into the display region and the non-display region may be applied to, not only the determination of whether or not the measurement point is to be displayed, but also to a case where the state of display is to be changed stepwise. In that case, the first determination condition includes stepwise determination criteria, and the first determination function 45b changes the state of display of the image in the display region according to determination results based on the stepwise determination criteria. For example, a plurality of thresholds may be set stepwise for the correlation coefficient results and the measurement points may be classified so that as the correlation coefficient decreases, the transparency in the color display increases (the point becoming more transparent). Furthermore, the first determination condition may be set to be a less restrictive condition so that all of the scan region is determined as the display region.

The spatial filter 45c performs filter processing in the spatial direction, such as smoothing filtering, on the strain values corresponding to the measurement points classified as the display region by the first determination function 45b. The persistence function 45d performs filter processing for temporal smoothing on data of the distortion values that have been subjected to the filter processing. The temporal filter processing is performed on a plurality of frames each of which has been subjected to the filter processing by the spatial filter 45c, and time-series data are thereby smoothed. The persistence function 45d outputs the data of the strain distribution that have been processed, to the image memory 46.

On the basis of a second determination condition having a determination criterion higher than that of the first determination condition with respect to the data and the parameter, the second determination function 45e determines a generation condition for the image. Specifically, on the basis of a second determination criterion having a determination criterion related to the signal intensities of the data and a determination criterion related to at least one of displacement and variance of a part included in the scan region and a correlation coefficient related to pattern matching of the part, the second determination criterion having a determination criterion stricter than the first determination condition, the second determination function 45e determines the generation condition for color display of strain elastography in the scan region.

That is, the second determination function 45e classifies the measurement points into measurement points to be used in strain value calculation (calculation of the average value and the maximum value of strain values) and measurement points not to be used in the strain value calculation, on the basis of the signal intensity at each measurement point of the data collected in the strain elastography mode and the displacement, variance, and correlation coefficient at each measurement point calculated by the Doppler processing circuitry 44, and calculates the average value, the maximum value, the minimum value, and the standard deviation, using the strain values at the measurement points to be used in the strain value calculation. The second determination function 45e outputs the calculated average value, maximum value, minimum value, and standard deviation, to the image memory 46.

For example, for each of the data and the parameter, the second determination condition has, as a determination criterion, a threshold based on an absolute value or a threshold based on a relative value. In this case, the second determination function 45e compares thresholds set for the signal intensity, displacement, correlation coefficient, and variance (variance of the displacement and variance of the correlation coefficient) with results at each measurement point and thereby determines a region to be used in strain value calculation (calculation of the average value and maximum value of strain values) and a region not to be used in the strain value calculation. The variance values of the Doppler data may be used as the variance. The second determination condition referred to by the second determination function 45e has a determination criterion stricter than that of the first determination condition.

For example, in a case where signal intensities are used for determination, the thresholds in the second determination condition are set higher than the thresholds in the first determination condition, and the second determination function 45e determines any measurement point having a signal intensity higher than the threshold in the second determination condition as a measurement point to be used in the strain value calculation. Two thresholds (third threshold>fourth threshold) may be set for the signal intensities. In this case, the two thresholds in the second determination condition may be set so that “first threshold>third threshold”, and “fourth threshold>second threshold”, in comparison with the two thresholds (first threshold>second threshold) in the first determination condition. The second determination function 45e may determine any measurement point having a signal intensity lower than the third threshold and higher than the fourth threshold, as a measurement point to be used in the strain value calculation.

In a case where displacement is used for determination, the threshold in the second determination condition is set lower than the threshold in the first determination condition, and the second determination function 45e determines any measurement point having higher displacement than the threshold as a measurement point not to be used in the strain value calculation, and any measurement point having displacement equal to or less than the threshold as a measurement point to be used in the strain value calculation.

In a case where variance is used for determination, the threshold in the second determination condition is set lower than the threshold in the first determination condition, and the second determination function 45e determines any measurement point having displacement variance, correlation coefficient variance, or Doppler variance higher than the threshold as a measurement point not to be used in the strain value calculation, and any measurement point having the variance equal to or less than the threshold as a measurement point to be used in the strain value calculation.

In a case where correlation coefficients are used for determination, the threshold in the second determination condition is set higher (for example, to 0.7) than the threshold (for example, 0.6) in the first determination condition, and the second determination function 45e determines any measurement point having a correlation coefficient equal to or less than the threshold as a measurement point not to be used in the strain value calculation and any measurement point having a correlation coefficient larger than the threshold as a measurement point to be used in the strain value calculation.

The second determination function 45e may determine measurement points to be used in the strain value calculation by using one of the above described determination criteria or may determine measurement points to be used in the strain value calculation by using a plurality of the above described determination criteria.

The second determination condition includes, similarly to the first determination condition, a determination criterion including a combination of the data and the parameter. Specifically, the second determination function 45e is able to determine measurement points to be used in the strain value calculation by combining the signal intensities, displacement, variance, and correlation coefficients. For example, the second determination function 45e may calculate a new parameter by an arithmetic operation using at least two or more of the signal intensity, displacement, variance, and correlation coefficient (for example, multiplication of the signal intensity and the displacement) and determine measurement points to be used in the strain value calculation by using the calculated new parameter. In this case also, a condition stricter than the first determination condition is set for the new parameter.

Furthermore, in a case where the first determination condition is the logical sum of the data and the parameter, the second determination condition includes a logical product of the data and the parameter, and the second determination function 45e determines a generation condition for the image by using the parameter related to elasticity corresponding to a region satisfying the condition of the logical product in the scan region. That is, the second determination function 45e executes, for each measurement point, at least two or more determinations of a determination based on a signal intensity, a determination based on displacement, and a determination based on a correlation coefficient, and in a case where a determination result indicating that the measurement point is a measurement point to be used in the strain value calculation is obtained in all of the determinations, the second determination function 45e determines that measurement point as a measurement point to be used in the strain value calculation.

For example, the second determination function 45e calculates a logical product of a determination based on a signal intensity and a determination based on a correlation coefficient, and classifies any measurement point determined as a measurement point to be used in the strain value calculation in both of these determinations as a measurement point to be used in the strain value calculation. In a case where the logical sum is used for the first determination condition and the logical product is used for the second determination condition, the threshold for the first determination condition and the threshold for the second determination condition may be the same.

The classification for the strain value calculation may be applied to, not only the determination of whether the measurement point is to be used or not to be used in the strain value calculation, but also a case where weighting of measurement points to be used in the strain value calculation is performed stepwise. In that case, the second determination condition includes stepwise determination criteria, and the second determination function 45e performs weighting for the parameter related to elasticity used in the determination of the generation condition for the image, according to determination results based on the stepwise determination criteria. For example, a plurality of thresholds may be set stepwise for the correlation coefficient, and weighting may be performed such that a weight applied to a distortion value increases as the correlation coefficient increases. That is, the second determination function 45e performs weighting such that a higher weight is applied to a strain value of a measurement point having a higher correlation coefficient and calculates, for example, an average value or a maximum value. In a case where all of the measurement points have been determined as a measurement point to be used in the strain value calculation in the determination using the second determination condition, the second determination function 45e determines the average value, maximum value, minimum value, and standard deviation using the strain values of all of the measurement points.

The normalization function 45f reads, for example, the strain distribution data that have been processed by the persistence function 45d and the average value, maximum value, minimum value, and standard deviation calculated by the second determination function 45e, from the image memory 46, performs normalization to assign distortion values to a color map, and thereby sets a color map LUT 45h.

The data interpolation function 45g performs data interpolation processing by scan-converting the strain distribution data that have been processed by the persistence function 45d into a data format for display. The data that have been scan-converted are data resulting from replacement of the strain values of the tissue at the measurement points with pixel values of pixels in the display image.

In accordance with the color map LUT 45h, the generation function 45i generates elasticity image data having colors assigned to the pixels in the data that have been scan-converted, the colors being in accordance with the pixel values (strain values). Furthermore, the generation function 45i generates B-mode image data in the scan region.

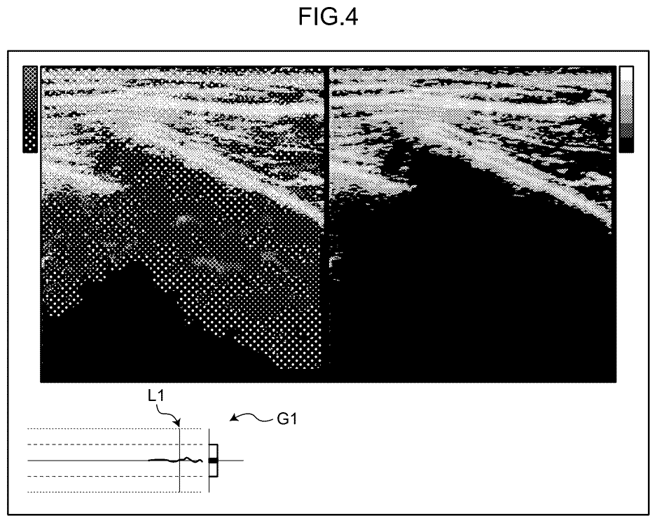

The control circuitry 48 causes the display 2 to display the image data generated by the generation function 45i. Specifically, the control circuitry 48 causes an image to be displayed, the image having been generated on the basis of a generation condition for a display region in the scan region. FIG. 4 is a diagram illustrating an example of display according to the first embodiment. For example, as illustrated in FIG. 4, the control circuitry 48 displays B-mode image data (on the right in FIG. 4) and composite image data (on the left in FIG. 4) having the B-mode image data and elasticity image data composited onto each other, side by side. As illustrated in FIG. 4, with the elasticity image data according to the embodiment, tissue to be actually observed (the upper region in the image) is able to be displayed in color on the basis of appropriate strain values. The control circuitry 48 enables display, as a moving image, of images based on the parameter related to elasticity, the images having been generated over time for the scan region. That is, the control circuitry 48 causes the moving image of the elasticity image data to be displayed by sequentially compositing the elasticity image data, which are sequentially generated by applying the above-described various processing to each set of ultrasound image data collected over time in the strain elastography mode, with the B-mode image data.

Furthermore, as illustrated in FIG. 4, the control circuitry 48 enables a graph G1 to be displayed further, the graph G1 representing temporal changes in the parameter related to elasticity during scanning of a subject. The graph G1 is a graph having the horizontal axis representing time and the vertical axis representing average velocity and represents temporal changes in average velocity of the tissue. As illustrated in FIG. 4, the tissue average velocity periodically alternates between positive and negative values as pressing and release of the observed part by means of the ultrasound probe 1 are repeated. Furthermore, in the graph G1, a line L1 is drawn at a position of a time corresponding to the elasticity image data that are currently being displayed. That is, in a case where the elasticity image data are being displayed as a moving image, the line L1 moves along the time axis (horizontal axis) of the graph G1.

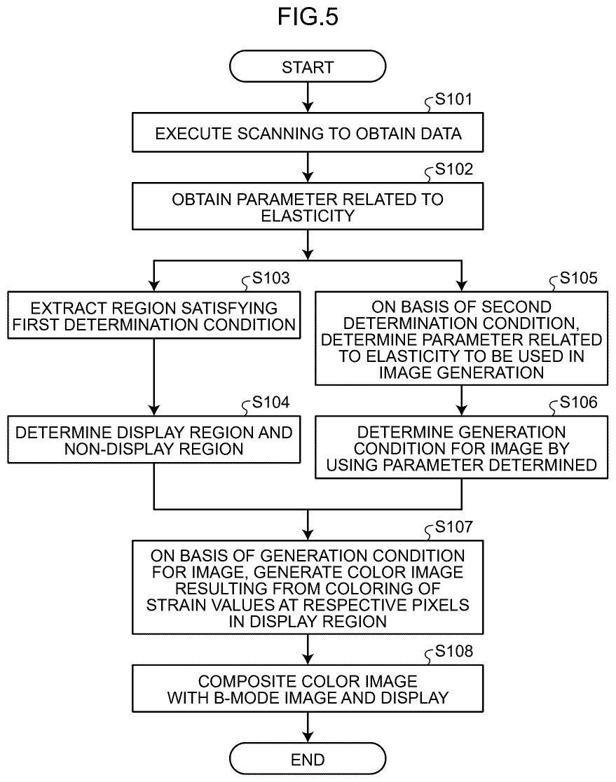

A procedure of processing at the ultrasound diagnostic apparatus 10 will be described next by use of FIG. 5. FIG. 5 is a flowchart illustrating a procedure of processing of the ultrasound diagnostic apparatus 10 according to the first embodiment.

For example, as illustrated in FIG. 5, in this embodiment, the control circuitry 48 executes scanning to obtain data (Step S101). Specifically, the control circuitry 48 obtains each set of data by executing B-mode scanning and strain elastography mode scanning of a scan region.

Subsequently, the Doppler processing circuitry 44 obtains a parameter related to elasticity using the data obtained by the strain elastography mode scanning (Step S102).

Subsequently, the image processing circuitry 45 extracts a region satisfying the first determination condition (Step S103) and determines a display region and a non-display region of elasticity image data based on the parameter related to elasticity (Step S104). Simultaneously, the image processing circuitry 45 determines, on the basis of the second determination condition, a parameter (strain value) related to elasticity used in image generation (Step S105), and determines a generation condition for an image (for example, the average value and maximum value of strain values) using the parameter determined (Step S106).

Subsequently, on the basis of the image generation condition, the image processing circuitry 45 generates a color image (elasticity image data) resulting from coloring of strain values at respective pixels in the display region (Step S107). On the basis of data (signal) obtained by B-mode scanning at Step S101, the image processing circuitry 45 generates B-mode image data. The control circuitry 48 composites the color image with the B-mode image and causes the resultant image to be displayed (Step S108).

As described above, according to the first embodiment, the Doppler processing circuitry 44, the first determination function 45b, the second determination function 45e, and the control circuitry 48 are included. The Doppler processing circuitry 44 calculates a parameter related to elasticity using data obtained by scanning of a subject. The first determination function 45b determines, on the basis of a first determination condition related to the data and the parameter, a display region and a non-display region for an image based on the parameter related to elasticity, in a scan region where the scanning has been executed. The second determination function 45e determines, on the basis of a second determination condition having a determination criterion higher than that of the first determination condition with respect to the data and the parameter, a generation condition for the image. The control circuitry 48 causes an image to be displayed, the image having been generated on the basis of the generation condition for the display region in the scan region. Therefore, the ultrasound diagnostic apparatus 10 according to the first embodiment determines a generation condition for elasticity image data by excluding inappropriate values from strain values, is able to generate the elasticity image data based on the generation condition for a region to be observed, and thus enables color display based on appropriate strain values for the region to be observed in an image.

Furthermore, according to the first embodiment, the first determination condition and the second determination condition each include a determination criterion that is a combination of the data and the parameter. Therefore, the ultrasound diagnostic apparatus 10 according to the first embodiment is able to use appropriate determination conditions.

Furthermore, according to the first embodiment, the combination of the data and the parameter includes at least one of an arithmetic operation using the data and the parameter and a logical operation using the data and the parameter. Therefore, the ultrasound diagnostic apparatus 10 according to the first embodiment is able to set various conditions according to statuses.

Furthermore, according to the first embodiment, the first determination condition includes stepwise determination criteria, and the first determination function 45b changes a display state of the image in the display region according to determination results based on the stepwise determination criteria. Therefore, the ultrasound diagnostic apparatus 10 according to the first embodiment is able to perform display reflecting differences in the parameter.

Furthermore, according to the first embodiment, the second determination condition includes stepwise determination criteria, and the second determination function 45e performs weighting of the parameter related to elasticity used in determination of the generation condition for the image, according to determination results based on the stepwise determination criteria. Therefore, the ultrasound diagnostic apparatus 10 according to the first embodiment is able to perform color display reflecting differences in the parameter.

Furthermore, according to the first embodiment, the first determination condition includes a logical sum of the data and the parameter, and the first determination function 45b determines a region satisfying a condition of the logical sum in the scan region as the display region of the image. The second determination condition includes a logical product of the data and the parameter, and the second determination function 45e determines the generation condition for the image by using the parameter related to elasticity corresponding to a region satisfying a condition of the logical product in the scan region. Therefore, the ultrasound diagnostic apparatus 10 according to the first embodiment enables the determination based on the first determination condition and the determination based on the second determination condition, more appropriately.

Furthermore, according to the first embodiment, the first determination condition has, as a determination criterion, a threshold based on an absolute value or a threshold based on a relative value, for each of the data and the parameter, and the second determination condition has, as a determination criterion, a threshold based on an absolute value or a threshold based on a relative value, for each of the data and the parameter. Therefore, the ultrasound diagnostic apparatus 10 according to the first embodiment enables various comparison.

Furthermore, according to the first embodiment, the control circuitry 48 causes a graph to be displayed further, the graph representing temporal changes in the parameter related to elasticity during the scanning of the subject. Therefore, the ultrasound diagnostic apparatus 10 enables reference information to be provided, the reference information being related to elasticity image data.

Furthermore, according to the first embodiment, the control circuitry 48 causes images to be displayed as a moving image, the images being based on the parameter related to elasticity and having been generated over time for the scan region. Therefore, the ultrasound diagnostic apparatus 10 according to the first embodiment enables color display as display of a moving image, the color display being based on appropriate strain values for the region to be observed in the image.

Furthermore, according to the first embodiment, the first determination condition and the second determination condition each have a determination criterion related to a signal intensity of the data and a determination criterion related to at least one of displacement and variance of a part included in the scan region and a correlation coefficient related to pattern matching of the part. Therefore, the ultrasound diagnostic apparatus 10 according to the first embodiment enables appropriate determination processing.

### Other Embodiments

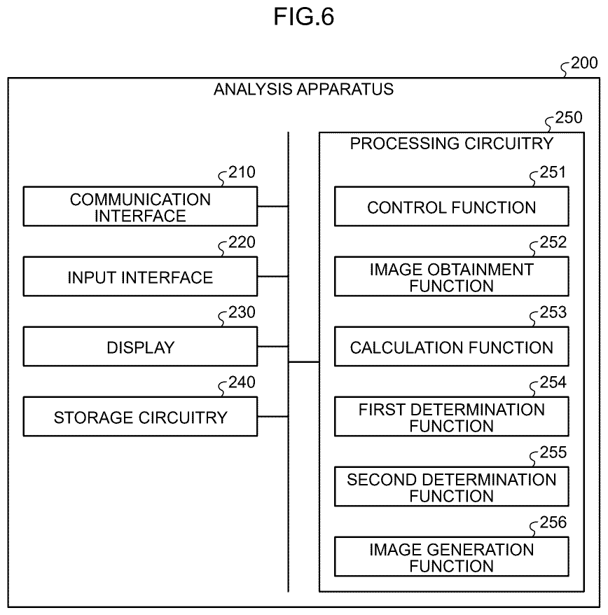

A case where the ultrasound diagnostic apparatus 10 executes various processing has been described with respect to the above described embodiment. However, the embodiment is not limited to this case and may be applied to a case where the various processing is performed by an analysis apparatus that is an apparatus external to the ultrasound diagnostic apparatus 10, for example. FIG. 6 is a block diagram illustrating an example of a configuration of an analysis apparatus 200 according to another embodiment. As illustrated in FIG. 6, the analysis apparatus 200 has a communication interface 210, an input interface 220, a display 230, storage circuitry 240, and processing circuitry 250. The analysis apparatus 200 is implemented by, for example, an information processing apparatus, such as a tablet terminal or a workstation.

The communication interface 210 is connected to the processing circuitry 250, and controls various data transmission and communication performed with a medical image diagnostic apparatus (for example, the ultrasound diagnostic apparatus 10) connected via a network not illustrated in the figure, or with an image storage apparatus, for example. For example, the communication interface 210 is implemented by a network card, a network adapter, or a network interface controller (NIC).

The input interface 220 is implemented by any of, for example, a trackball, a switch button, a mouse, a keyboard, a touch pad with an operation surface touched for input operation, a touch monitor having a display screen and a touch pad integrated together, a non-contact input circuit using an optical sensor, and a sound input circuit, which are for performing various setting. The input interface 220 is connected to the processing circuitry 250, converts input operation received from an operator, into an electric signal, and outputs the converted electric signal to the processing circuitry 250. According to this specification, the input interface 220 is not necessarily an input interface including physical operation parts, such as a mouse and a keyboard. For example, examples of an input interface also include a processing circuit that receives an electric signal corresponding to input operation from an external input device provided separately from the apparatus and outputs this electric signal to a control circuit.

The display 230 is connected to the processing circuitry 250 and displays various information and various images output from the processing circuitry 250. For example, the display 230 is implemented by a liquid crystal monitor, a cathode ray tube (CRT) monitor, or a touch monitor. For example, the display 230 displays a user interface (UI) for receiving an instruction from an operator, various images, and various processing results obtained by the processing circuitry 250.

The storage circuitry 240 is connected to the processing circuitry 250 and stores various data. For example, the storage circuitry 240 is implemented by: a semiconductor memory element, such as a random access memory (RAM) or a flash memory; a hard disk; and/or an optical disk. For example, the storage circuitry 240 stores ultrasound images. Furthermore, the storage circuitry 240 stores various information used in processing performed by the processing circuitry 250 and processing results obtained by the processing circuitry 250.

According to input operation received from an operator via the input interface 220, the processing circuitry 250 controls each component included in the analysis apparatus 200. The processing circuitry 250 causes the storage circuitry 240 to store a medical image output from the communication interface 210. Furthermore, the processing circuitry 250 reads a medical image from the storage circuitry 240 and causes the display 230 to display the read medical image.

As illustrated in FIG. 6, the processing circuitry 250 executes, for example, a control function 251, an image obtainment function 252, a calculation function 253, a first determination function 254, a second determination function 255, and an image generation function 256. For example, processing functions executed by the control function 251, the image obtainment function 252, the calculation function 253, the first determination function 254, the second determination function 255, and the image generation function 256, which are components of the processing circuitry 250 illustrated in FIG. 6, are recorded in the storage circuitry 240 in the form of programs that are able to be executed by a computer. The processing circuitry 250 is, for example, a processor, reads these programs from the storage circuitry 240, and implements the functions corresponding to the read programs by executing the read programs. In other words, the processing circuitry 250 that has read the programs has the functions illustrated in the processing circuitry 250 in FIG. 6.

The control function 251 performs overall control of the analysis apparatus 200. Furthermore, the control function 251 executes processing similar to that of the control circuitry 48 described above with respect to image display. The image obtainment function 252 obtains ultrasound image data collected by the ultrasound diagnostic apparatus 10 (data collected by B-mode scanning and data collected by strain elastography mode scanning) via a network not illustrated in the figure. The calculation function 253 executes processing similar to that of the Doppler processing circuitry 44 described above. The first determination function 254 executes processing similar to that of the first determination function 45b described above. The second determination function 255 executes processing similar to that of the second determination function 45e described above. The image generation function 256 executes processing similar to that of the generation function 45i described above.

The term, “processor”, used in above the description means, for example: a central processing unit (CPU); a graphics processing unit (GPU); or a circuit, such as an application specific integrated circuit (ASIC) or a programmable logic device (for example, a simple programmable logic device (SPLD), a complex programmable logic device (CPLD), or a field programmable gate array (FPGA)). The processor implements functions by reading and executing programs stored in a memory. Instead of being stored in the memory, the programs may be directly incorporated in a circuit of the processor. In this case, the processor implements the functions by reading and executing the programs incorporated in the circuit. Each processor according to the embodiment is not necessarily configured as a single circuit, and a plurality of independent circuits may be combined together to be configured as a single processor to implement the functions.

The components of each apparatus are functionally and conceptually illustrated in the drawings for the embodiment described above and are not necessarily configured physically as illustrated in the drawings. That is, specific forms of separation and integration of each apparatus are not limited to those illustrated in the drawings, and all or part thereof may be configured by functional or physical separation or integration in any units according to various loads and use situations. In addition, all or any part of the processing functions executed in each apparatus may be implemented by a CPU and a program analyzed and executed by the CPU, or implemented as hardware by wired logic.

Furthermore, the method described above with respect to the embodiment may be implemented by a computer executing a program prepared beforehand, the computer being, for example, a personal computer or a workstation. This program may be distributed via a network, such as the Internet. Furthermore, the program may be recorded in a non-transitory recording medium readable by a computer, such as a hard disk, a flexible disk (FD), a CD-ROM, an MO, a DVD, a USB memory, or a flash memory, such as an SD card memory, and executed by being read from the non-transitory recording medium by a computer.

As described above, an embodiment enables color display based on appropriate strain values for a region to be observed in an image.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An analysis apparatus, comprising:
processing circuitry configured to
calculate a parameter related to elasticity using data obtained by scanning of a subject;
determine, on the basis of a first determination condition related to the data and the parameter, a display region and a non-display region for an image based on the parameter related to elasticity, in a scan region where the scanning has been executed;
determine, on the basis of a second determination condition having a determination criterion higher than that of the first determination condition with respect to the data and the parameter, a generation condition for the image; and
cause an image to be displayed, the image having been generated on the basis of the generation condition for the display region in the scan region.

2. The analysis apparatus according to claim 1, wherein the first determination condition and the second determination condition each include a determination criterion that is a combination of the data and the parameter.

3. The analysis apparatus according to claim 2, wherein the combination of the data and the parameter includes at least one of an arithmetic operation using the data and the parameter and a logical operation using the data and the parameter.

4. The analysis apparatus according to claim 1, wherein
the first determination condition includes stepwise determination criteria, and
the processing circuitry is configured to change a display state of the image in the display region, according to determination results based on the stepwise determination criteria.

5. The analysis apparatus according to claim 1, wherein
the second determination condition includes stepwise determination criteria, and
the processing circuitry is configured to perform weighting of the parameter related to elasticity used in determination of the generation condition for the image, according to determination results based on the stepwise determination criteria.

6. The analysis apparatus according to claim 3, wherein
the first determination condition includes a logical sum of the data and the parameter, and
the processing circuitry is configured to determine a region satisfying a condition of the logical sum in the scan region as the display region of the image.

7. The analysis apparatus according to claim 3, wherein
the second determination condition includes a logical product of the data and the parameter, and
the processing circuitry is configured to determine the generation condition for the image by using the parameter related to elasticity corresponding to a region satisfying a condition of the logical product in the scan region.

8. The analysis apparatus according to claim 6, wherein
the second determination condition includes a logical product of the data and the parameter, and
the processing circuitry is configured to determine the generation condition for the image by using the parameter related to elasticity corresponding to a region satisfying a condition of the logical product in the scan region.

9. The analysis apparatus according to claim 1, wherein
the first determination condition has, as a determination criterion, a threshold based on an absolute value or a threshold based on a relative value, for each of the data and the parameter, and
the second determination condition has, as a determination criterion, a threshold based on an absolute value or a threshold based on a relative value, for each of the data and the parameter.

10. The analysis apparatus according to claim 1, wherein the processing circuitry is configured to cause a graph to be displayed further, the graph representing temporal changes in the parameter related to elasticity during the scanning of the subject.

11. The analysis apparatus according to claim 1, wherein the processing circuitry is configured to cause images to be displayed as a moving image, the images being based on the parameter related to elasticity and having been generated over time for the scan region.

12. The analysis apparatus according to claim 1, wherein the first determination condition and the second determination condition each have a determination criterion related to a signal intensity of the data, and a determination criterion related to at least one of displacement and variance of a part included in the scan region and a correlation coefficient related to pattern matching of the part.

13. An ultrasound diagnostic apparatus, comprising:
processing circuitry configured to
obtain data by scanning of a subject;
calculate a parameter related to elasticity using the data;
determine, on the basis of a first determination condition related to the data and the parameter, a display region and a non-display region for an image based on the parameter related to elasticity, in a scan region where the scanning has been executed;
determine, on the basis of a second determination condition having a determination criterion higher than that of the first determination condition with respect to the data and the parameter, a generation condition for the image; and
cause an image to be displayed, the image having been generated on the basis of the generation condition for the display region in the scan region.

14. A non-transitory storage medium storing a program that causes a computer to execute the steps of:
calculating a parameter related to elasticity using data obtained by scanning of a subject;
determining, on the basis of a first determination condition related to the data and the parameter, a display region and a non-display region for an image based on the parameter related to elasticity, in a scan region where the scanning has been executed;
determining, on the basis of a second determination condition having a determination criterion higher than that of the first determination condition with respect to the data and the parameter, a generation condition for the image; and
causing an image to be displayed, the image having been generated on the basis of the generation condition for the display region in the scan region.
